SEQUENCE LISTING (2 sequences)

SEQ ID NO: 1 - DNA, 21 bp, Artificial
  siRNA sequence
aatctagcggaggtaaagaag

SEQ ID NO: 2 - DNA, 21 bp, Artificial
  siRNA sequence
aagacctaatcgtgggactgc

(19) United States
(12) Patent Application Publication
(10) Pub. No.: US 2010/0292169 A1
(43) Pub. Date: Nov. 18, 2010

(54) METHODS AND COMPOSITIONS FOR REGULATING HDAC6 ACTIVITY

(75) Inventors: Tso-Pang Yao, Chapel Hill, NC (US); Jeffrey J. Kovacs, Durham, NC (US); Charlotte Hubbert, Seattle, WA (US); Yi-shan Lee, Chapel Hill, NC (US); Ya-sheng Gao, Chapel Hill, NC (US); June-Tai Wu, Banciao City (TW)

Correspondence Address: MYERS BIGEL SIBLEY & SAJOVEC, PO BOX 37428, RALEIGH, NC, 27627, US

(73) Assignee: Duke University

(21) Appl. No.: 12/700,832

(22) Filed: Feb. 5, 2010

Related U.S. Application Data
(63) Continuation of application No. 11/643,295, filed on Dec. 21, 2006, now abandoned.
(60) Provisional application No. 60/752,611, filed on Dec. 21, 2005.

(51) Int. Cl. A61K 38/12; A61K 31/16; A61K 31/7088; A61P 35/00; A61P 3/10; A61P 17/14; C12N 5/02

(52) U.S. Cl. 514/21.1; 514/507; 514/44 A; 435/375

(57) ABSTRACT

The present invention provides methods and compositions for inhibiting Hsp90 activity in a cell, comprising contacting the cell with an inhibitor of histone deacetylase 6 (HDAC6)

### PRIORITY STATEMENT

This application is a continuation application of, and claims priority to, U.S. application Ser. No. 11/643,295, filed Dec. 21, 2006 (abandoned), which claims the benefit, under 35 U.S.C. §119(e), of U.S. Provisional Application No. 60/752,611, filed Dec. 21, 2005, the entire contents of each of which are incorporated by reference herein.

### STATEMENT OF GOVERNMENT SUPPORT

This invention was supported in part by funding under Government Grant No. W81XWH-04-1-0555, awarded by the Department of Defense. The United States Government has certain rights in this invention.

### BACKGROUND OF THE INVENTION

The heat shock protein Hsp90 and its co-factors form molecular chaperone complexes that facilitate the structural maturation of its substrates, termed client proteins. The Hsp90-assisted maturation of client proteins often leads to an enhanced activity and stability. Prominent examples of Hsp90 client proteins include steroid hormone receptors and kinases important for oncogenesis (Richter and Buchner, 2001). Among them, the Hsp90-dependent maturation of glucocorticoid receptor (GR), a member of the steroid hormone receptor family, is best characterized. GR mediates biological effects of glucocorticoid by acting as a transcription factor (Giguere et al., 1986). Upon binding to glucocorticoid, GR becomes activated and translocates into the nucleus where it controls specific transcriptional programs. In the absence of its ligand, however, GR is inactive and resides in the cytoplasm where it associates with Hsp90 (Cadepond et al., 1991). It has been shown that the association with Hsp90 is critical for GR to assume a competent ligand-binding conformation. In vitro and in vivo analyses demonstrate that Hsp90, in conjunction with a selected set of co-chaperone proteins, is required for GR to bind hormone with high affinity (Pratt and Toft, 2003). The study of Hsp90-dependent GR maturation has provided mechanistic insight into the basic steps of chaperone complex-client protein assembly and the important functions of co-chaperones (Dittmar et al., 1997). However, the critical question regarding whether and how Hsp90 is regulated in these processes is poorly understood.

The characterization of the deacetylase HDAC6, a member of the histone deacetylase family, has implicated protein acetylation in the regulation of microtubules, growth factor-induced chemotaxis and the processing of misfolded protein aggregates (Haggarty et al., 2003; Hubbert et al., 2002; Kawaguchi et al., 2003; Matsuyama et al., 2002; Zhang et al., 2003). Consistent with these apparently non-genomic functions, HDAC6 is mainly localized to the cytoplasm (Hubbert et al., 2002; Verdel et al., 2000).

The present invention is based on studies that demonstrate that Hsp90 is a substrate of HDAC6 and that its chaperone activity is regulated by acetylation. Thus, the present invention provides methods and compositions for modulating Hsp90 activity and modulating steroid receptor signaling in a cell by modulating HDAC6 activity.

### SUMMARY OF THE INVENTION

The present invention provides a method of inhibiting Hsp90 activity in a cell, comprising contacting the cell with an inhibitor of histone deacetylase 6 (HDAC6).

Further provided herein is a method of treating a cancer associated with Hsp90 in a subject, comprising administering to the subject an effective amount of an inhibitor of HDAC6.

In addition, the present invention provides a method of modulating steroid receptor signaling in a cell, comprising contacting the cell with an inhibitor of HDAC6.

Also provided herein is a method of treating a disorder associated with aberrant steroid receptor signaling in a subject, comprising administering to the subject an effective amount of an inhibitor of HDAC6.

### DETAILED DESCRIPTION OF THE INVENTION

As used herein, “a” or “an” or “the” can mean one or more than one. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is based on the unexpected discovery that Hsp90 activity can be modulated by altering HDAC6 activity. This, in one embodiment provided herein, the present invention provides a method of inhibiting Hsp90 activity in a cell, comprising contacting the cell with an inhibitor of histone deacetylase 6 (HDAC6) activity.

In other embodiments, the present invention provides a method of enhancing HDAC6 activity. Such enhancement of HDAC6 activity can be used e.g., in a method of treating a neurodegenerative disease and/or diabetes, by protecting against cell death caused by misfolded protein accumulation.

The present invention further provides a method of treating a cancer associated with Hsp90 activity in a subject, comprising administering to the subject an effective amount of an inhibitor of HDAC6 activity. A cancer associated with Hsp90 activity of this invention can be any cancer associated with an oncoprotein. As used herein, an oncoprotein means a protein encoded by an oncogene. An oncogene as used herein is a gene that produces a gene product that can potentially induce neoplastic transformation of a cell. Nonlimiting examples of oncogenes include genes for growth factors, growth factor receptors, protein kinases, signal transducers, nuclear phosphoproteins, and transcription factors. When these genes are constitutively expressed after structural and/or regulatory changes in a cell, uncontrolled cell proliferation can result. An oncogene can have a viral or cellular origin. Viral oncogenes generally have the prefix “v-” before the gene symbol; cellular oncogenes (e.g., proto-oncogenes) have the prefix “c-” before the gene symbol.

Nonlimiting examples of an oncoprotein of this invention include ErbB2 (Her2/Neu), EGFR/ErbB 1, ErbB3, ErbB4, ErbB5 and any other erbB family members, PDGFR, PML-RAR AKT, BCR-abl, src, Raf family members (e.g., C-Raf, B-Raf), dominant negative p53, HIF-1α, Telomerase, MTG8 (myeloid leukemia protein), Heat Shock factor, Hepatitis B virus reverse transcriptase, c-src, v-src, mutated or absent p53, Hsp70, estrogen receptor, mutant K-ras proteins, nitric oxide synthase and chimeric protein710 pBCR-ABL individually and/or in any combination. The present invention further includes any other oncoprotein now known or later identified to be associated with Hsp90.

A cancer associated with an oncoprotein of this invention can be, but is not limited to, B cell lymphoma, T cell lymphoma, myeloma, leukemia (AML, ALL, CML, CLL), hematopoietic neoplasias, thymoma, lymphoma, sarcoma, lung cancer, liver cancer, non-Hodgkins lymphoma, Hodgkins lymphoma, Burkitt's lymphoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, sebaceous cell carcinoma, brain cancer (astrocytoma, glioma, glioblastoma, ependymoma, medulloblastoma, meningioma, oligodendroglioma, oligoastrocytoma), angiosarcoma, hemangiosarcoma, adenocarcinoma, liposarcoma, head and neck carcinoma, thyroid carcinoma, soft tissue sarcoma, osteosarcoma, testicular cancer, uterine cancer, cervical cancer, gastrointestinal cancer, colorectal cancer, oral cancer, nasopharyngeal cancer, oropharyngeal cancer, esophageal cancer, stomach cancer, multiple myeloma, bile duct cancer, cervical cancer, laryngeal cancer, penile cancer, urethral cancer, anal cancer, vulvar cancer, vaginal cancer, gall bladder cancer, thymoma, salivary gland cancer, lip and oral cavity cancer, adenocortical cancer, non-melanoma skin cancer, pleura mesothelioma, joint cancer, hypopharyngeal cancer, ureter cancer, peritoneum cancer, omentum cancer, mesentery cancer, Ewing's sarcoma, rhabdomyosarcoma, spinal cord cancer, endometrial cancer, neuroblastoma, pituitary cancer, retinoblastoma, eye cancer, islet cell cancer, and any other cancer now known or later identified to be associated with an oncoprotein and/or associated with Hsp90 activity and/or associated with HDAC6 activity.

In further aspects, the present invention provides a method of modulating steroid receptor signaling in a cell, comprising contacting the cell with an inhibitor of HDAC6 activity.

As used herein, “steroid hormone receptor” means an intracellular (typically cytoplasmic) receptor that perform signal transduction for steroid hormones. “Signal transduction” or “signaling” as used herein describes a set of chemical reactions in a cell that occurs when a molecule, such as a hormone, attaches to a receptor. The signal transduction is a cascade of biochemical reactions inside the cell that eventually reach the target molecule or reaction. Thus, signal transduction or signaling as used herein is a method by which molecules inside the cell can be altered by molecules outside the cell.

A steroid receptor of this invention can include but is not limited to a glucocorticoid receptor, an androgen receptor (AR), an estrogen receptor (ER), a progesterone receptor (PR), a mineralcorticoid receptor (MR), a retinoid acid receptor, a Vitamin D receptor, a thyroid hormone receptor, and/or any other steroid receptor now known or later identified, the signaling of which can be inhibited by inhibiting the activity of Hsp90 and/or inhibiting the activity of HDAC6.

Further provided herein is a method of treating a disorder associated with aberrant steroid receptor signaling in a subject, comprising administering to the subject an effective amount of an inhibitor of HDAC6 activity. As used herein, aberrant steroid receptor signaling″ means that a receptor becomes hyperactive or hypoactive and is usually caused by mutation(s) within the receptor gene itself (including promoters) and/or by mutations affecting genes that regulate receptor activity. Aberrant signaling can also describe receptor activity in cells in a diseased state, such as cancer, where receptor activity plays a role in promoting the disease state.

A disorder of this invention that is associated with aberrant steroid receptor signaling can be but is not limited to a cancer of this invention as described herein (e.g., prostate cancer associated with mutations and/or overexpression of AR; breast cancer associated with ER activity), muscle atrophy (e.g., disuse atrophy, cachexia; caused by increased levels of GR), type II diabetes (e.g., caused by aberrant GR signaling resulting in GR-induced gluconeogenesis and increased blood sugar), polycystic ovarian syndrome (e.g., caused by aberrant androgen receptor signaling, resulting in hyperandrogenism), male pattern baldness (e.g., caused by aberrant androgen receptor signaling), uterine fibroids, endometriosis (e.g., caused by aberrant progesterone receptor signaling).

A cell of this invention can be any cell with Hsp90 activity and HDAC6 activity. Such a cell can be in vitro, ex vivo and/or in vivo. Nonlimiting examples of a cell line of this invention include A549 cells, 293 cells, 293T cells, SKBR3 cells, MCF7 cells and A431 cells.

A cell of this invention can be a cell in a subject of this invention and such a cell can be any cell in the subject with Hsp90 activity and HDAC6 activity. A subject of this invention can be any animal that produces Hsp90 and HDAC6. Nonlimiting examples of a subject of this invention include mammals such as humans, mice, dogs, cats, horses, cows, rabbits, goats, etc.

The methods of the present invention employ an inhibitor of HDAC6 activity. An inhibitor of HDAC6 activity is any compound, agent or material that has an inhibitory effect on the activity of HDAC6. An inhibitory effect means that the amount of activity of HDAC6 that is measured in an assay in the absence of an HDAC6 inhibitor is reduced when the inhibitor is added to the assay. Assays to measure HDAC6 activity are known in the art and some of these assays are described in the EXAMPLES section included herewith.

An inhibitor of HDAC6 activity of this invention can be but is not limited to hydroxamic acid based HDAC inhibitors, Suberoylanilide hydroxamic acid (SAHA) and its derivatives, NVP-LAQ824, Trichostatin A, Scriptaid, m-Carboxycinnamic acid bishydroxamic acid (CBHA), ABHA, Pyroxamide, Propenamides, Oxamflatin, 6-(3-Chlorophenylureido)caproic hydroxamic acid (3-C1-UCHA), A-161906, jnj 16241199, tubacin and tubacin analogs, siRNA (e.g., SEQ ID NO:1; SEQ ID NO:2; siRNA 194-214), short chain fatty acid HDAC inhibitors, butyrate, phenylbutyrate, sodium butyrate, valproate, (−)-Depudecin, Sirtinol, hydroxamic acid, trichostatins, epoxyketone-containing cyclic tetrapeptides, trapoxins, HC-toxin, Chlamydocin, Diheteropeptide, WF-3161, Cyl-1, Cyl-2, non-epoxyketone-containing cyclic tetrapeptides, PXD101, dimeric HDAC inhibitors, depsipeptide, FR901228 (FK228), Apicidin, APHA Compound 8, cyclic-hydroxamic-acid-containing peptides (CHAPS), benzamides and benzamide analogs, MS-275 (MS-27-275), CI-994, LBH589, deprudecin, organosulfur compounds and any combination thereof. In some embodiments of this invention, the HDAC6 inhibitor is tubacin, either alone or in any combination with an inhibitor of HDAC6 activity of this invention. It is also contemplated in some embodiments that one or more than one inhibitor of HDAC6 is excluded from the list of inhibitors of HDAC6 of this invention.

An inhibitor of HDAC6 activity that can be employed in the methods of this invention can be an inhibitor that acts at the level of transcription and/or translation of the HDAC6 protein, whereby such an inhibitor alters HDAC6 activity by decreasing the amount of functional HDAC6 protein produced. An inhibitor of HDAC6 activity can be, but is not limited to, an antisense nucleic acid, a ribozyme (e.g., as described in U.S. Pat. No. 5,877,022), RNAs that effect spliceosome-mediated trans-splicing (Puttaraju et al. (1999) Nature Biotech. 17:246; U.S. Pat. No. 6,013,487; U.S. Pat. No. 6,083,702), RNAs that trigger RNA interference mechanisms (RNAi), including small interfering RNAs (siRNA) that mediate gene silencing (Kawaguchi et al., (2003) “The deacetylase HDAC6 regulates aggresome formation and cell viability in response to misfolded protein stress” Cell 115:727-738; Sharp et al. (2000) Science 287:2431) and/or other non-translated RNAs, such as “guide” RNAs (Gorman et al., (1998) Proc. Nat. Acad. Sci. USA 95:4929; U.S. Pat. No. 5,869,248 to Yuan et al.), and the like, as are known in the art. These transcription/translation inhibitors can be employed in the methods of this invention individually, in combination with one another and/or in combination with other HDAC6 inhibitors of this invention. A nonlimiting example of a siRNA (also termed shRNA) has the nucleotide sequence AATCTAGCGGAGGTAAAGAAG (SEQ ID NO:1) and AAGACCTAATCGTGGGACTGC (SEQ ID NO:2). The production and identification of additional siRNA sequences that can be employed in the methods of this invention are well known in the art and thus one of skill in the art would be able to readily produce any number of additional siRNA sequences based on the known nucleotide sequence for HDAC6 and test each such sequence for activity as a silencing RNA of HDAC6, according to standard methods in the art. Thus, the present invention includes any siRNA of HDAC6, the production and characterization of which is well within the skill of the ordinary artisan.

Methods of the present invention can further include a method of regulating Hsp70 activity (e.g., degrading misfolded proteins) by regulating HDAC6 activity. For example, the degradation of misfolded proteins can be inhibited by inhibiting Hsp70 at the level of inhibition of HDAC6 activity. By regulating Hsp70 activity (e.g., by inhibiting Hsp70 activity by introducing an HDAC6 inhibitor) cancer cells can be targeted for death, due to the accumulation, rather than the degradation, of misfolded proteins, in cancers where the accumulation of misfolded proteins is toxic to cancer cells.

The present invention further provides methods of identifying a substance as a modulator of HDAC6 activity. Thus, in some embodiments, the present invention provides a method of identifying a substance as an inhibitor of HDAC6 activity comprising; a) contacting the substance with HDAC6 and a substrate that is deacetylated by HDAC6, under conditions whereby the deacetylation activity of HDAC6 can occur and measuring the amount of deacetylation of the substrate by HDAC6 in the presence of the substance; b) measuring the amount of deacetylation of the substrate in the absence of the substance; and c) comparing the amount of deacetylation of the substrate of step (a) and step (b), whereby a decrease in the amount of deactylation of the substrate of step (a) identifies a substance as an inhibitor of HDAC6 activity.

Further provided is a method of identifying a substance as an inhibitor of HDAC6 activity comprising; a) contacting the substance with HDAC6 and a substrate that is deacetylated by HDAC6, under conditions whereby the deacetylation activity of HDAC6 can occur and measuring the amount of acetylation of the substrate in the presence of the substance; b) measuring the amount of acetylation of the substrate in the absence of the substance; and c) comparing the amount of acetylation of the substrate of step (a) and step (b), whereby an increase in the amount of acetylation of the substrate of step (a) identifies a substance as an inhibitor of HDAC6 activity.

In addition, the present invention provides a identifying a substance as an enhancer of HDAC6 activity comprising; a) contacting the substance with HDAC6 and a substrate that is deacetylated by HDAC6, under conditions whereby the deacetylation activity of HDAC6 can occur and measuring the amount of deacetylation of the substrate by HDAC6 in the presence of the substance; b) measuring the amount of deacetylation of the substrate in the absence of the substance; and c) comparing the amount of deacetylation of the substrate of step (a) and step (b), whereby an increase in the amount of deactylation of step (a) identifies a substance as an enhancer of HDAC6 activity.

Further provided is a method of identifying a substance as an enhancer of HDAC6 activity comprising: a) contacting the substance with HDAC6 and a substrate that is deacetylated by HDAC6, under conditions whereby the deacetylation activity of HDAC6 can occur and measuring the amount of acetylation of the substrate in the presence of the substance; b) measuring the amount of acetylation of the substrate in the absence of the substance; and d) comparing the amount of acetylation of the substrate of step (a) and step (b), whereby a decrease in the amount of acetylation of the substrate of step (a) identifies a substance as an enhancer of HDAC6 activity.

In the screening methods described herein, the measurement of the amount of deacetylation of a substrate or of the amount of acetylation of a substrate is carried out according to methods standard in the art, such as, e.g., the methods described in the EXAMPLES section herein. Nonlimiting examples of assays that can be carried out to measure HDAC6 activity according to the methods of this invention include a tubulin deacetylation assay (Hubbert et al. (2002) “HDAC6 is a microtubule-associated deacetylase” Nature 417:455-458) and commercially-available fluorescence-based assays as are known in the art.

In some embodiments, the methods of this invention can include steps comprising the administration, along with the administration of an inhibitor of HDAC6 activity to a subject, of an effective amount of one or more therapeutic agents and/or therapeutic treatments to treat cancer and/or a disorder caused by aberrant hormone receptor signaling. These therapeutic agents and/or treatments can be administered before, after and/or simultaneously with the administration of the inhibitor of HDAC6 activity. Furthermore, as noted herein, the HDAC6 activity inhibitor of this invention can be combined in a single composition with one or more therapeutic agent(s) and/or treatment(s) of this invention and/or maintained in a single composition that can be administered in combination with other single compositions comprising therapeutic agent(s) of this invention, and such combined administration of agents and/or treatments can be simultaneous and/or in any order.

Non-limiting examples of therapeutic agents and treatments that can be used in combination with an HDAC6 inhibitor according to the methods of this invention include inhibitors of Hsp90 (e.g., geldanamycin), chemotherapeutic drugs and reagents (e.g., docetaxel, paclitaxel, carboplatin), proteasome inhibitors (e.g., Hideshima et al. (2005) “Small-molecule inhibition of proteasome and aggresome function induces synergistic antitumor activity in multiple myeloma” PNAS USA 102:8567-8572), microtubule-targeting agents (e.g., taxol; see Marcus et al. (2005) “The synergistic combination of the farnesyl transferase inhibitor lonafarnib and paclitaxel enhances tubulin acetylation and requires a functional tubulin deacetylase” Cancer Research 65:3883-3893), treatments that induce misfolded protein accumulation, including those that generate oxidative stress (e.g., radiation), etc. as would be known to the skilled artisan.

As used herein, “effective amount” refers to an amount of a compound or composition of this invention that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

“Treat,” “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

The present invention further provides a composition (e.g., a pharmaceutical composition) comprising an inhibitor of HDAC6 activity, either alone (e.g., as a single HDAC6 inhibitor or as a single composition of one or more HDAC6 inhibitors) and/or in any combination with one or more therapeutic reagents of this invention (e.g., a chemotherapeutic drug, an Hsp90 inhibitor, etc.), and these compositions can be present in a pharmaceutically acceptable carrier. The compositions described herein can be formulated for administration in a pharmaceutical carrier in accordance with known techniques. See, e.g. Remington, The Science And Practice of Pharmacy (latest edition). By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., bbth skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 0.1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis/tris buffer (pH 6) or ethanol/water and can contain from 0.1 to 0.2M active ingredient.

An effective amount of a composition of this invention, the use of which is in the scope of present invention, will vary from composition to composition, and subject to subject, and will depend upon a variety of well known factors such as the age and condition of the patient and the form of the composition and route of delivery. An effective amount can be determined in accordance with routine pharmacological procedures known to those skilled in the art. As a general proposition, a dosage from about 0.1 μg/kg to about 50 mg/kg will have therapeutic efficacy, with all weights being calculated based upon the weight of the composition.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

The compositions of this invention can be administered to a cell of a subject either in vivo or ex vivo. For administration to a cell of the subject in vivo, as well as for administration to the subject, the compositions of this invention can be administered, for example as noted above, orally, parenterally intravenously), by intramuscular injection, intradermally (e.g., by gene gun), by intraperitoneal injection, subcutaneous injection, transdermally, extracorporeally, topically or the like.

If ex vivo methods are employed, cells or tissues can be removed and maintained outside the body according to standard protocols well known in the art while the compositions of this invention are introduced into the cells or tissues. For example, a nucleic acid of this invention can be introduced into cells via any gene transfer mechanism, such as, for example, virus-mediated gene delivery, calcium phosphate mediated gene delivery, electroporation, microinjection and/or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

The present invention is more particularly described in the Examples set forth below, which are not intended to be limiting of the embodiments of this invention.

### EXAMPLES

### Example 1

### HDAC6 Regulates Hsp90 Acetylation and Chaperone-Dependent Activation of Glucocorticoid Receptor

Cell lines. A549 and NIH-3T3 cell lines overexpressing HDAC6 wild type, ΔBUZ or catalytically inactive mutants were established using retroviral infection. A549 and 293T cells stably expressing siRNA for HDAC6 were established as described previously (Kawaguchi et al., 2003).

Antibodies. Rabbit polyclonal HDAC6 antibody DU227 was raised against a C-terminal HDAC6 peptide as described previously (Hubbert et al., 2002). The production of antibodies for acetylated lysine (Komatsu et al., 2003), Hsp90 (H1090) and p23 (JJ3) has been described (Johnson and Toft, 1994). GR antibody was purchased from Cell Signaling. S-14 antibody recognizing HDAC6 was purchased from Santa Cruz.

Immunoprecipitation and immunostaining. Cells were lysed as described previously (Hubbert et al., 2002). Hsp90 antibody was pre-incubated with rabbit-anti-mouse (Jackson Labs) and Protein-A Sepharose beads (Roche) for 10 minutes. The bead/antibody mix was added to 750 μg of whole cell lysate and incubated at 4° C. for 3 hours. Samples were washed 4 times with 150 mM NETN(Hubbert et al., 2002) and subjected to SDS-PAGE and immunoblotting analysis. Immunolocalization of GR and HDAC6 was described previously (Hubbert et al., 2002).

Ligand Binding Assay. 293T cells stably transfected with HDAC6 siRNA or control (pSuper) plasmid were lysed in 1.5 volumes of buffer (10 mM Hepes, pH 7.35, 1 mM EDTA, 20 mM Na₂MoO₄) and centrifuged at 100,000 g. Aliquots (150 μl) of cytosol were incubated overnight at 4° C. with 100 nM [³H]dexamethasone plus or minus a 1.000-fold excess of non-radioactive dexamethasone. Free steroid was removed with dextran-coated charcoal, and steroid binding was expressed as cpm of [³H]dexamethasone/100 pJ of cell cytosol, +/−SEM for three experiments with assays performed in triplicate.

HDAC6 associates with Hsp90 in vivo. Using an affinity trap approach, Hsp90 was identified as a prominent HDAC6 interacting partner by both mass spectrometry and direct immunoprecipitation, which shows that endogenous HDAC6 and Hsp90 can be abundantly and specifically co-immunoprecipitated from multiple cell lines (e.g., A431 cells and A549 cells were immunoprecipitated using cc-HDAC6 antibody or pre-immune serum (PI), and immunoblotted for Hsp90).

To further characterize the HDAC6-Hsp90 interaction, additional assessments were carried out to determine whether mutations or pharmacological inhibitors that affect HDAC6 activity would influence its association with Hsp90. Full HDAC6 function requires both its deacetylase activity and ubiquitin-binding activity, which is mediated by a unique zinc finger, termed the BUZ finger (Hubbert et al., 2002; Kawaguchi et al. 2003). Inactivation of HDAC6 either by mutations or by the inhibitor, trichostatin A (TSA), treatment was shown to lead to the dissociation of HDAC6 from Hsp90. Furthermore, an HDAC6 mutant lacking the ubiquitin-binding BUZ finger also failed to bind Hsp90 efficiently [e.g., cell lysates from NIH-3T3 cell lines stably overexpressing Flag-HDAC6, Flag-HDAC6-ΔBUZ (ubiquitin-binding deficient mutant), Flag-HDAC6-cat-mut (H216/611A, catalytically inactive mutant), or Neo vector control were immunoprecipitated using α-FLAG antibody and blotted with α-Hsp90; A549 cells were left untreated or subjected to a 4 hour treatment of trichostatin A (1 μM) and cell lysates were immunoprecipitated with α-HDAC6 antibody and blotted for Hsp90]. These data demonstrate that the HDAC6-Hsp90 interaction is specific and requires both deacetylase and ubiquitin-binding activities of HDAC6.

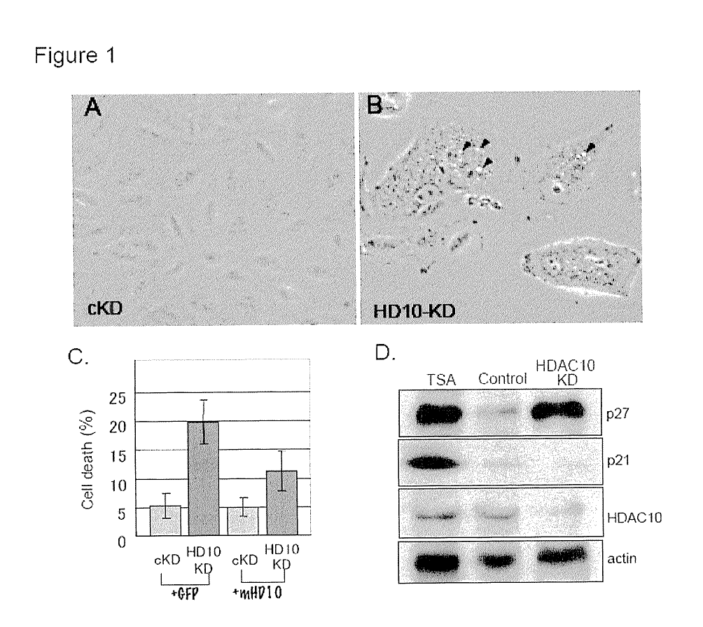

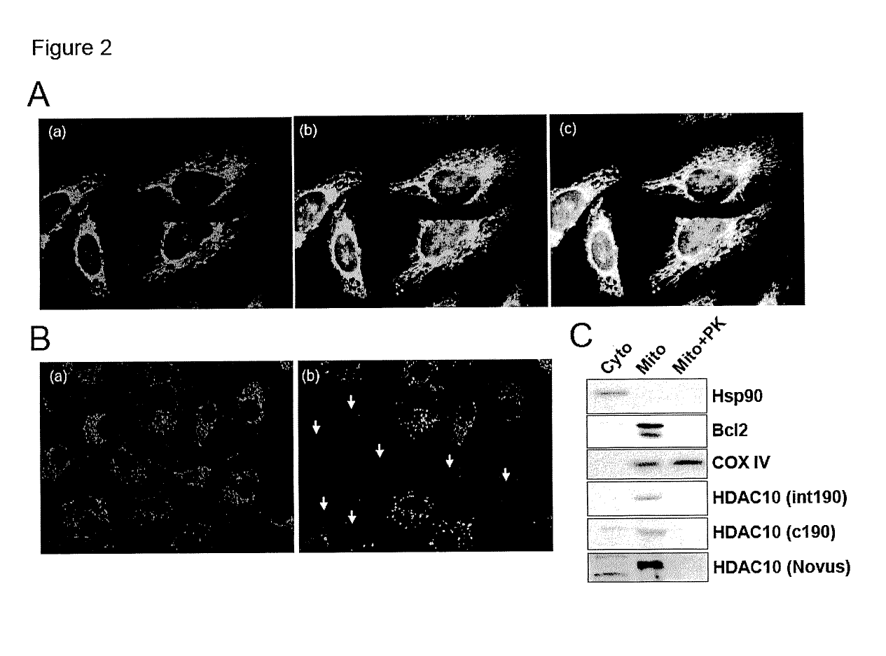

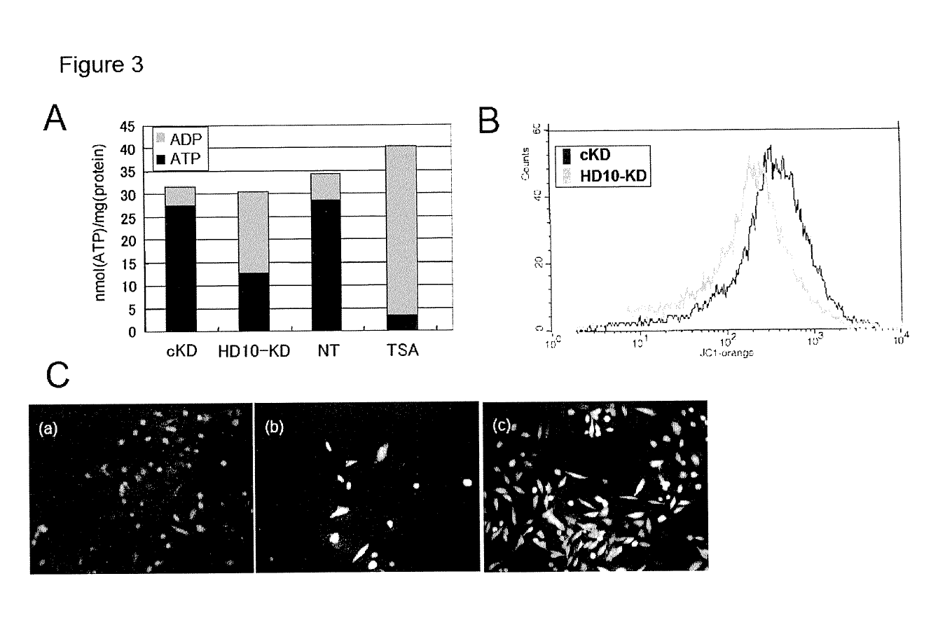

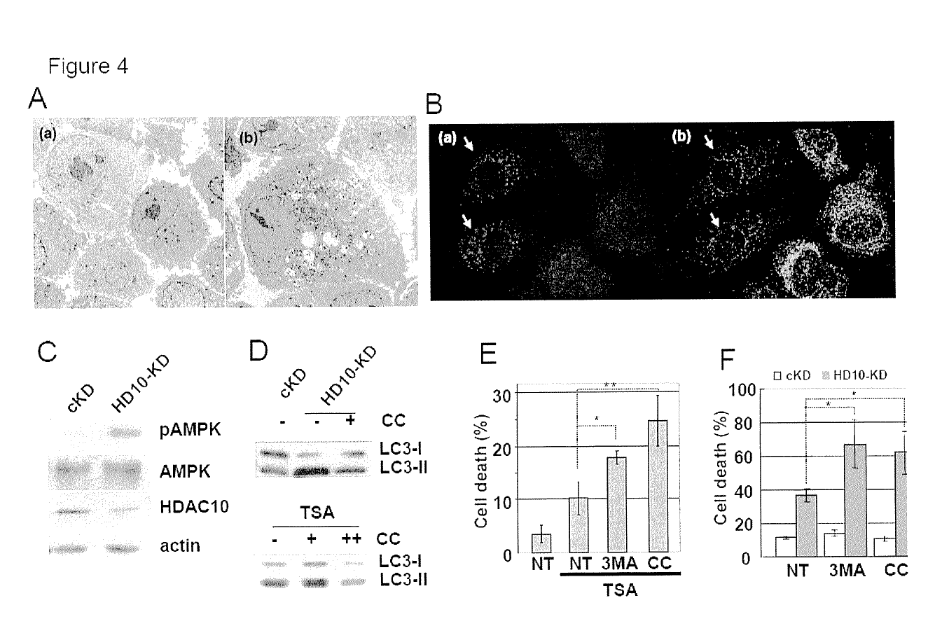

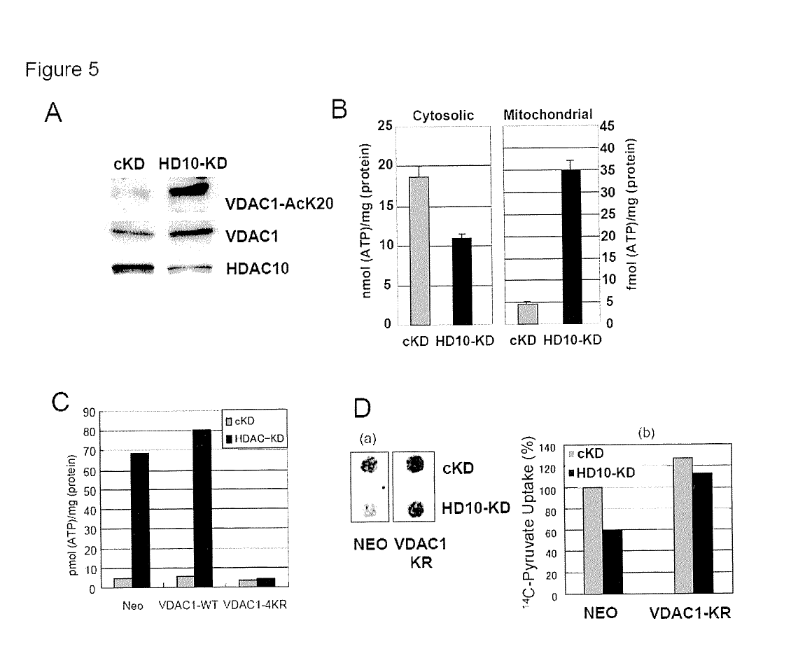

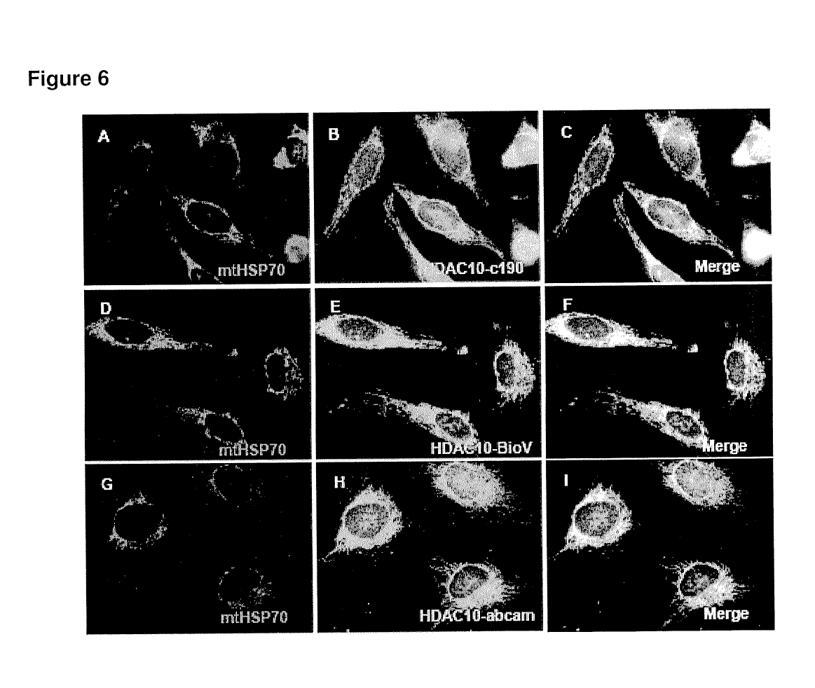

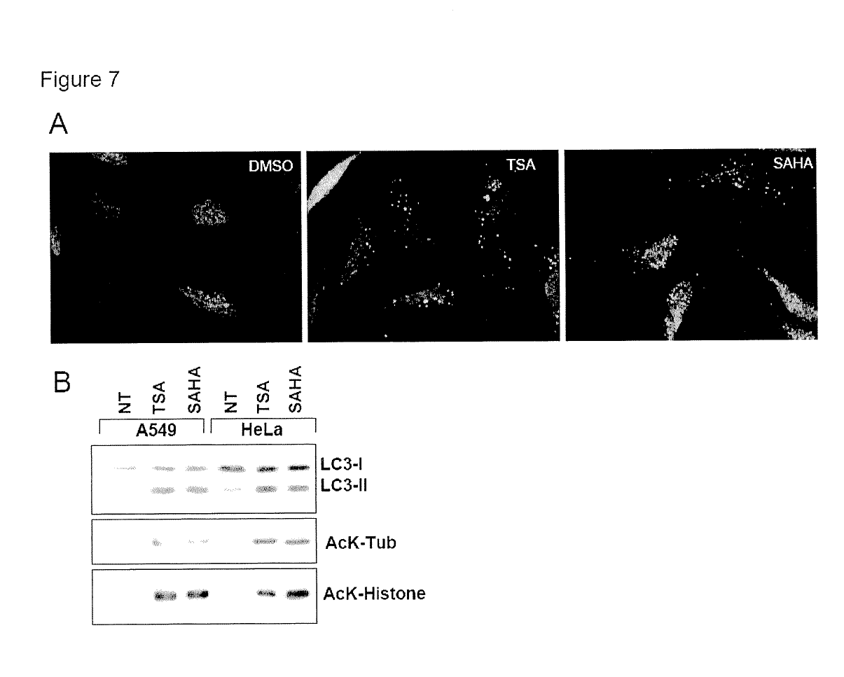

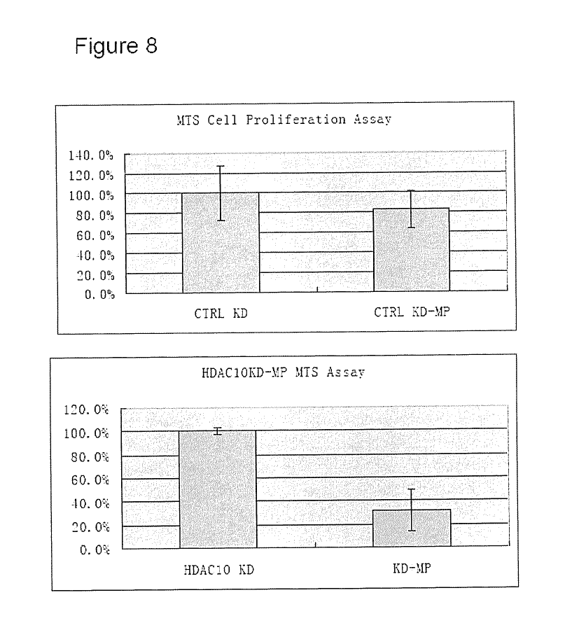

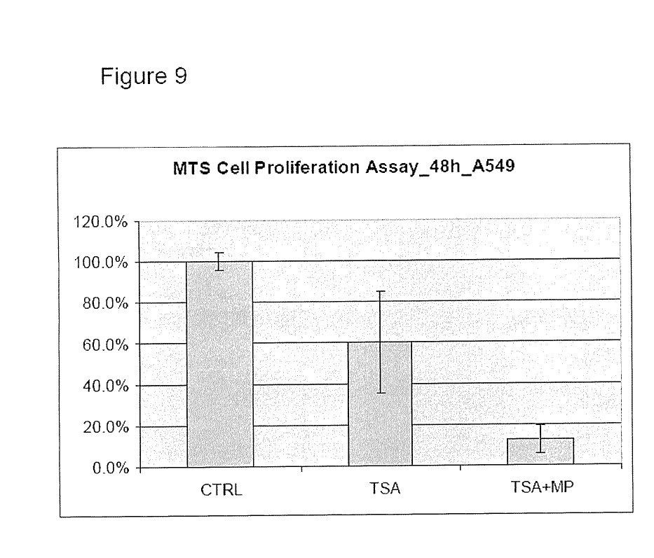

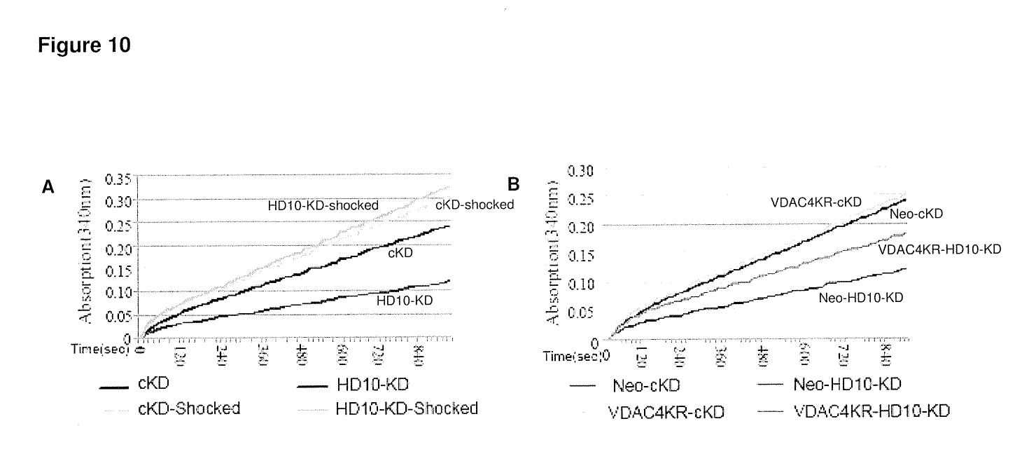

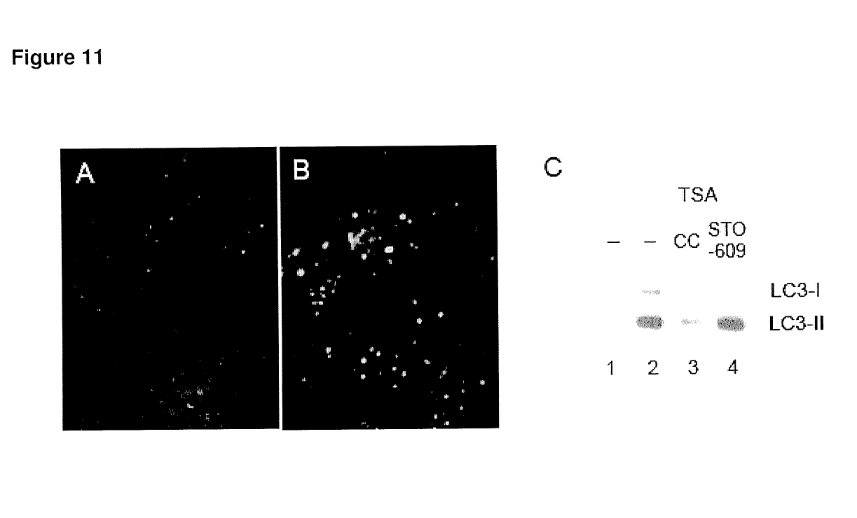

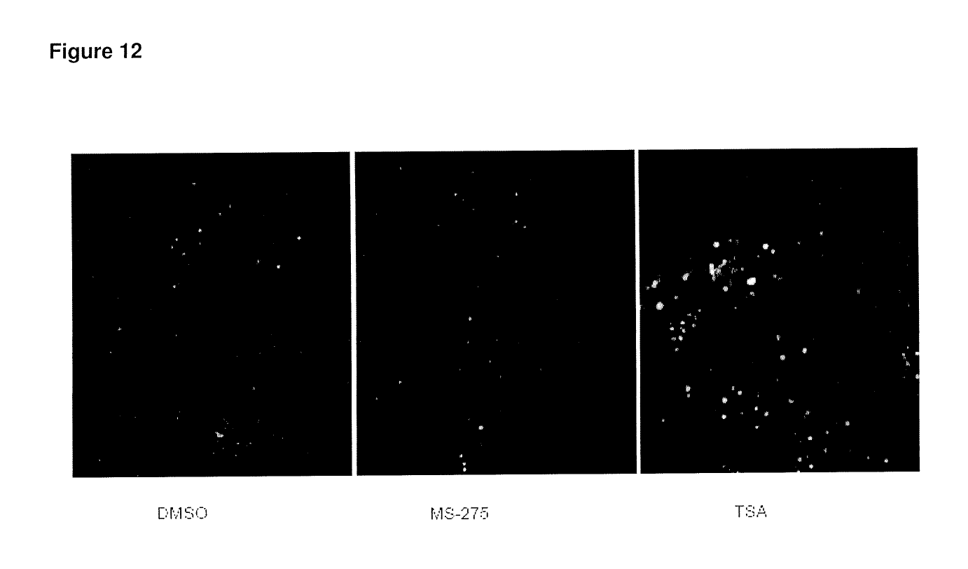

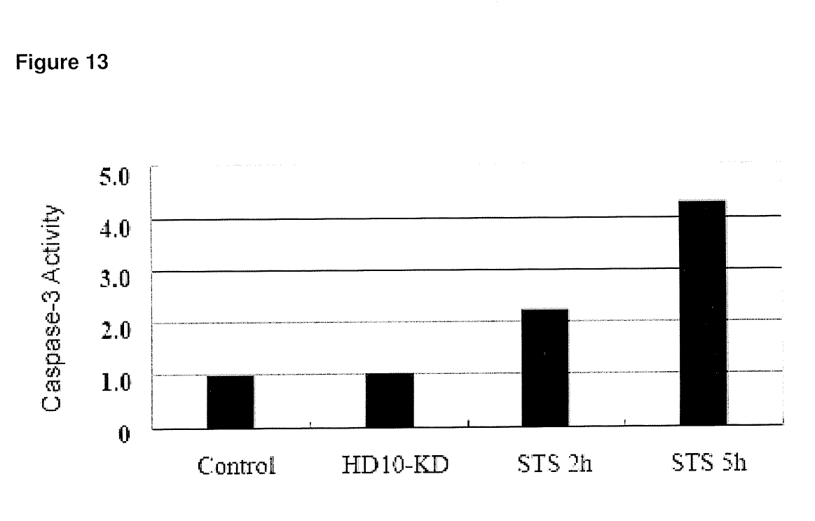

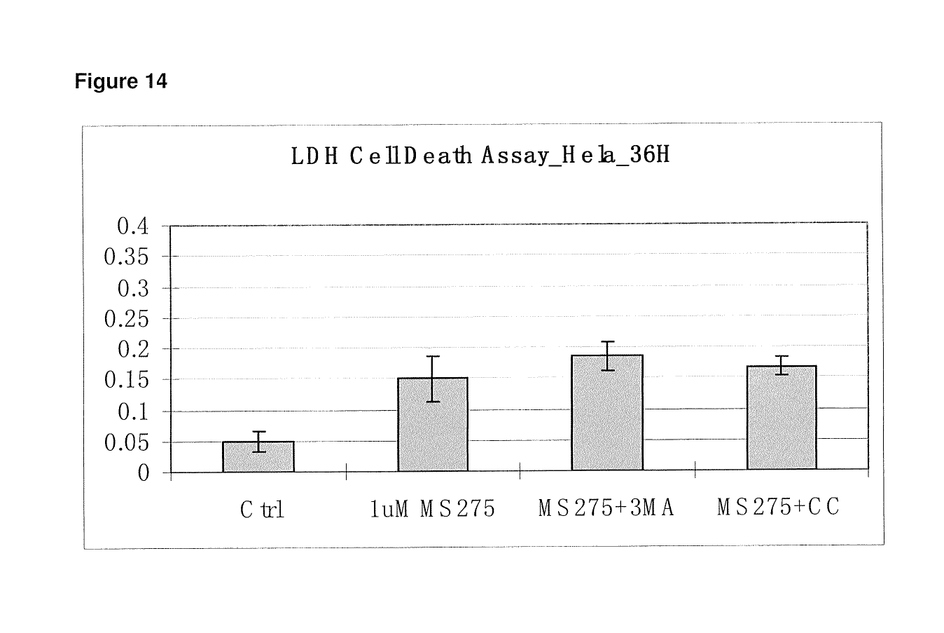

HDAC6 regulates Hsp90 acetylation. To investigate the possibility that HDAC6 functions as an Hsp90 deacetylase, a determination was made regarding whether over-expression of HDAC6 can lead to Hsp90 deacetylation in vivo. Lysates from A549 cell lines stably overexpressing wild type (wt), catalytically inactive mutant HDAC6 (cat-mut) or Neo vector control were immunoprecipitated with α-Hsp90 and then immunoblotted with anti-acetylated lysine antibody (α-AcK). A basal level of Hsp90 acetylation could be detected in control A549 cell lines. Hsp90 acetylation levels, however, were markedly reduced in A549 cells that stably over-express wild type but not a catalytically inactive mutant HDAC6. Conversely, in A549 cells stably expressing siRNA that reduces HDAC6 expression [HDAC6 knockdown (Kawaguchi et al., 2003)], Hsp90 acetylation levels are substantially increased (e.g., A549 cells stably expressing pSuper control plasmid (wt) or HDAC6 siRNA (KD) were left untreated, treated for 4 hours with TSA, or treated for 4 hours with TPXb (100 nM). Cell lysates were then immunoprecipitated with α-Hsp90 followed by immunoblotting with α-AcK antibody. Both HDAC6 siRNA and TSA treatment cause a dramatic rise in the level of acetylated Hsp90). These results show that HDAC6 can function as an Hsp90 deacetylase in vivo. Additionally, it was found that TSA but not TPXb, a potent inhibitor for all HDAC members except HDAC6 (Furumai et al., 2001), also induces potent Hsp90 acetylation (e.g., A549 cells were treated with TSA for 8 hours to induce an acetylated population of Hsp90. Hsp90 was immunoprecipitated from cell lysates and immunoblotted with α-AcK to show an enrichment of acetylated Hsp90 (Input). 293T cells were transfected with FLAG-tagged wild-type or catalytically dead HDAC6. These cells were lysed and wt or cat-dead HDAC6 was immunprecipitated using α-FLAG antibody. The purified Hsp90 was then incubated with the purified FLAG-HDAC6 wt or cat-dead protein at 37° C. for 60 minutes. The reactions were then subjected to SDS-PAGE and immunoblotted with the indicated antibodies). It was noted that TSA treatment has little effect on Hsp90 acetylation in HDAC6 knockdown cells, indicating that HDAC6 is the primary TSA-sensitive endogenous Hsp90 deacetylase. Importantly, immuno-purified wild type but not catalytically inactive mutant HDAC6 can efficiently deacetylate acetylated Hsp90 in vitro as well (FIG. 2C). Together, these results demonstrate that HDAC6 has Hsp90 deacetylase activity.

Chaperone-dependent GR maturation is defective in HDAC6 deficient cells. Studies were conducted to determine if HDAC6-regulated Hsp90 acetylation is important for Hsp90 chaperone function. The requirement of Hsp90 chaperone activity for efficient ligand binding and the subsequent activation and nuclear translocation of the glucocorticoid receptor (GR) is the most well characterized function for Hsp90. To establish whether acetylation is important for Hsp90-dependent GR ligand binding, cytosols prepared from control and HDAC6 knockdown 293T cells were incubated with radiolabeled dexamethasone, and steroid binding to the GR was determined. Endogenous GR from control 293T cells binds ³H-dexamethasone significantly. However, a dramatic decrease in steroid ligand binding activity was observed in HDAC6 knockdown 293T cells. Although comparable amounts of GR are present in cytosols from both cell types, GR from HDAC6 knockdown cells exhibited approximately a six-fold reduction in ligand binding activity, relative to control cells. This result demonstrates that GRs produced in HDAC6 knockdown cells are defective in ligand binding activity, indicating an Hsp90 chaperone deficiency associated with Hsp90 hyperacetylation.

A transcriptional reporter assay was carried out to examine the functional status of GR. In control cells, endogenous GR efficiently induced a glucocorticoid-responsive element (GRE)-driven luciferase reporter following addition of dexamethasone for 4-6 hours (e.g., control or HDAC6 knockdown 293T cells were transiently transfected with an MMTV-GRE-luciferase reporter with or without expression plasmids of wild type, catalytically inactive (cat-mut) or ΔBUZ mutant HDAC6. These plasmids contain silent mutations in the sequences targeted by siRNA for HDAC6. The transfection of a wt-HDAC6 restored GR transcriptional activity in HDAC6 KD cells. Relative luciferase activity was measured after a four hour treatment with dexamethasone and normalized to an internal control (β-galactosidase). In contrast, the same dexamethasone treatment only weakly activated the GR reporter in HDAC6 knockdown cells. A similar conclusion was reached with prolonged ligand treatment but with a less marked difference. Importantly, re-introduction of an siRNA-resistant plasmid expressing wild type HDAC6 (Kawaguchi et al., 2003) fully restored GR transcriptional activity in the HDAC6 knockdown cells, whereas the catalytically-inactive or BUZ finger-deletion mutant HDAC6 were ineffective, consistent with observations that these mutants do not bind Hsp90 and cannot deacetylate Hsp90 efficiently. To further establish that HDAC6 is required for optimal GR activity, an investigation was done on endogenous GR-target gene induction in response to dexamethasone stimulation. Messenger RNA levels of GR and IkB at 4 hours after dexamethasone stimulation were determined by quantitative RT-PCR and normalized to the mRNA levels of 36B4. A similar defect in endogenous GR-target gene induction by ligand was observed in HDAC6 knockdown cells. The observed defect in transcriptional activity in HDAC6 knockdown cells is mirrored by the loss of ligand-induced nuclear accumulation of GR. In control A549 cells, GR becomes almost exclusively localized to the nucleus within 30 minutes of ligand treatment. In contrast, GR remains in the cytoplasm in the substantial majority (˜80%) of HDAC6 knockdown cells following ligand addition (e.g., control and HDAC6 knockdown A549 cells were cultured in hormone free media for 24 hours and then stimulated with dexamethasone for 30 minutes. The localization of GR was determined by immunostaining with an oc-OR antibody. Immunofluorescence microscopy revealed that GR shows a pan-cell staining before dexamethasone treatment in both cell types. After dexamethasone treatment, GR efficiently translocates into the nucleus in control, but not in HDAC6 KD cells). Thus, Hsp90-dependent GR ligand binding, nuclear translocation and transcriptional activity are all defective in HDAC6 knockdown cells. Together, these results indicate that HDAC6-mediated deacetylation is required for Hsp90 chaperone function to activate GR.

Hsp90 hyperacetylation is induced by dexamethasone and correlated with the dissociation of functional chaperone-OR complexes. Experiments were conducted to determine whether Hsp90 acetylation is regulated by dexamethasone. Control and HDAC6 knockdown A549 cells were cultured in hormone free media for 24 hours, and then stimulated with dexamethasone for the indicated time in hours. Cell lysates were then immunoprecipitated with α-Hsp90 followed by immunoblotting with α-AcK antibody. The results of these experiments showed that in control cells, dexmethasone treatment did not have a marked effect. However, in HDAC6 knockdown cells, an increase of acetylation by dexmethasone was evident. These results are consistent with the idea that Hsp90 acetylation is induced upon dexamethasone treatment and this acetylation is efficiently removed by HDAC6.

Studies were also conducted to identify the molecular basis for the regulation of Hsp90 function via HDAC6-mediated deacetylation. Because the proper folding of GR by Hsp90 depends on the association of Hsp90 with a distinct set of co-chaperones into a chaperone complex (Neckers, 2002; Pratt and Toft, 2003), a determination was made regarding whether Hsp90 acetylation affects Hsp90/co-chaperone assembly. The p23 protein (Johnson and Toft, 1994) is a co-chaperone that stabilizes the Hsp90-GR complex and is critical for GR ligand binding activity in vitro and in vivo (Dittmar et al., 1997; Morishima et al., 2003). Control A549 cells and HDAC6 siRNA knockdown cells (KD) were left untreated or treated for 4 hours with TSA or TPXb. Lysates were immunoprecipitated with α-Hsp90 antibody followed by immunoblotting with α-p23 antibody. Co-immunoprecipitation assays demonstrated that Hsp90 associates with p23 in control cells. However, Hsp90-p23 interactions were dramatically reduced in HDAC6 knockdown cells. TSA treatment, which induces Hsp90 hyperacetylation, also disrupted Hsp90-p23 interactions. Conversely, TPXb treatment, which does not inhibit HDAC6 activity, had little effect. These results indicate that Hsp90 acetylation leads to the dissociation of p23 from Hsp90. As p23 is known to stabilize the Hsp90-GR complex (Dittmar et al., 1997), studies were conducted to determine whether acetylation affects Hsp90-GR complex formation. Lysates from control A549 cells or HDAC6 knockdown cells (KD) untreated or treated with TSA (4 hours) were immunoprecipitated with α-Hsp90 antibody followed by immunoblotting with α-GR antibody. The Hsp90 and GR interaction is significantly reduced in HDAC6 knockdown cells or by treatment with TSA, providing a plausible mechanism for the observed GR defects. These results show that loss of HDAC6 activity leads to Hsp90 hyperacetylation, disassembly of the Hsp90 chaperone complex, and dissociation of the client protein OR.

In summary, in this study, HDAC6-regulated reversible acetylation has been identified as an important mechanism that controls Hsp90 molecular chaperone function.

Hsp90 chaperone complexes stabilize client proteins and, in the case of GR, promote a conformation that allows efficient ligand binding and subsequent nuclear translocation and transcriptional activation. The present invention demonstrates that GR produced in HDAC6 deficient model cell lines is defective in all three activities, strongly indicating a defect in Hsp90 chaperone function. The ligand-binding defects of GR in HDAC6 knockdown cells can be rescued by Hsp90 purified from wild type cells in vitro. We found that Hsp90-p23 chaperone complex formation and the chaperone-client (Hsp90-GR) association are both compromised in HDAC6 knockdown cells. The accumulation of hyperacetylated Hsp90 in HDAC6 deficient cells indicates that acetylation negatively regulates Hsp90 function by lowering Hsp90 affinity for the critical co-chaperone p23. Thus, stable complexes with client proteins, such as GR, are not formed, resulting in a failure of client protein maturation.

### Example II

### Regulation of the Dynamics of Hsp90 Action on the Glucocorticoid Receptor by Acetylation/Deacetylation of the Chaperone

Untreated rabbit reticulocyte lysate was purchased from Green Hectares (Oregon, Wis.). [6,7-³H]Dexamethasone (40 Ci/mmol), [ring-3,5-³H] chloramphenicol (38 Ci/mmol), and ¹²⁵I-conjugated goat anti-mouse and goat anti-rabbit IgGs were obtained from Perkin Elmer Life Sciences (Boston, Mass.). Protein A-Sepharose, non-radioactive dexamethasone, trichostatin A, goat anti-mouse and goat anti-rabbit horseradish peroxidase-conjugated antibodies, and M2 monoclonal anti-FLAG IgG were from Sigma. Dulbecco's modified Eagle's medium was from Bio-Whittaker (Walkersville, Md.). The BuGR2 monoclonal IgG used to immunoblot the mouse GR, and the rabbit polyclonal antibody used to immunoblot human GR were from Affinity Bioreagents (Golden, Colo.). The AC88 monoclonal IgG used to immunoblot hsp90 was from StressGen Biotechnologies (Victoria, BC, Canada). The JJ3 monoclonal IgG used to immunoblot p23 was provided by Dr. David Toft (Mayo Clinic, Rochester, Minn.). The FiGR monoclonal IgG used to immunoadsorb the mouse GR was provided by Dr. Jack Bodwell (Dartmouth Medical School, Lebanon, N.H.), and the 8D3 monoclonal IgM used to immunoadsorb hsp90 was provided by Dr. Gary Perdew (Pennsylvania State University, University Park, Pa.). The pSV2Wrec plasmid encoding full length mouse GR and the mouse mammary tumor virus-chloramphenicol acetyltransferase (MMTV-CAT) reporter plasmid were provided by Dr. Edwin Sanchez (Medical College of Ohio, Toledo, Ohio). 293T human embryonic kidney cells stably expressing a pSuper control siRNA (293T-wt) or HDAC6 siRNA (293T-HDAC6 KD) were described previously (8). The expression plasmid pcDNA3-FLAG-tagged HDAC6 and rabbit antisera used to immunoblot HDAC6 (α-HDAC6) and acetylated lysine (α-AcK) were generated in the Yao laboratory and have been described (7,8).

Cell Culture and Cytosol Preparation—293T cells were cultured in Dulbecco's modified Eagle's medium (DMEM) supplemented with 10% bovine calf serum. Cells were harvested by scraping into Hanks' buffered saline solution and centrifugation. Cell pellets were washed in Hanks' buffered saline solution, resuspended in 1.5 volumes of HEM buffer (10 mM NaOH-Hepes, 1 mM EDTA, and 20 mM sodium molybdate, pH 7.4) with 1 μM trichostatin A (TSA), 1 mM phenylmethylsulfonyl fluoride and 1 tablet of Complete-Mini protease inhibitor mixture (Roche Applied Science, Penzberg, Germany) per 3 ml buffer, and ruptured by Dounce homogenization. The lysate was then centrifuged at 100,000× g for 30 min, and the supernatant, referred to as “cytosol” was collected, aliquoted, flash-frozen, and stored at −70° C. Mouse GR was expressed in Sf9 cells and cytosol was prepared as previously described (16).

Transient Transfection of mouse GR and MMTV-CAT Reporter. 293T cells were grown as monolayer cultures in 162 cm² culture flasks to ˜50% confluency, washed, and incubated with 5 ml serum-free medium containing 25 μg of plasmid DNA and 75 μl of TRANSFAST transfection reagent (Promega). After 1 h, 10 ml of DMEM with 10% bovine calf serum was added and the incubations were continued for 48 h. For transfection of MMTV-CAT reporter, wild-type and knockdown cells were grown as monolayer cultures in 35-mm culture wells to ˜50% confluency, washed, and incubated for 1 h with 1 ml serum-free medium containing 5 μg of plasmid DNA and 15 μl of TransFast transfection reagent. The transfection medium was replaced with regular medium, and the cells were incubated for 48 h. During the incubation, cells were treated for 20 h with various concentrations of dexamethasone.

Immunoadsorption of GR—Receptors were immunoadsorbed from aliquots of 50 μl (for measuring steroid binding) or 100 μl (for Western blotting) of Sf9 cell cytosol by rotation for 2 h at 4° C. with 18 μl of protein A-Sepharose precoupled to 10 μl of FiGR ascites suspended in 200 μl of TEG (10 mM TES, pH 7.6, 50 mM NaC1, 4 mM EDTA, 10% glycerol). Immunoadsorbed GR was stripped of endogenously associated hsp90 by incubating the immunopellet for an additional 2 h at 4° C. with 350 μl of 0.5 M NaCl in TEG buffer. The pellets were then washed once with 1 ml of TEG buffer followed by a second wash with 1 ml of Hepes buffer (10 mM Hepes, pH 7.4).

GR.hsp90 Heterocomplex Reconstitution. Immunopellets containing GR stripped of chaperones were incubated with 50 μl of reticulocyte lysate or 293T cell cytosol and 5 μl of an ATP-regenerating system (50 mM ATP, 250 mM creatine phosphate, 20 mM magnesium acetate, and 100 units/ml creatine phosphokinase). For heterocomplex reconstitution with purified proteins, immunopellets containing, stripped GR were incubated with 15 μg of ATP-agarose-purified knockdown or wild-type HEK293 hsp90, 15 μg of purified rabbit hsp70, 0.6 μg purified human Hop, 6 μg of purified human p23, 0.125 μg of purified YDJ-1 adjusted to 55 μl with HKD buffer (10 mM Hepes, pH 7.4, 100 mM KCl, 5 mM dithiothreitol) containing 20 mM sodium molybdate and 5 μl of the ATP-regenerating system. The assay mixtures were incubated for 20 min at 30° C. with suspension of the pellets by shaking the tubes every 2 min. At the end of the incubation, the pellets were washed twice with 1 ml of ice-cold TEGM buffer (TEG with 20 mM sodium molybdate) and assayed for steroid binding capacity and for GR-associated hsp90. The five-protein mixture containing purified knockdown hsp90 was incubated for 5 min at 30° C. with an α-FLAG immune pellet prepared from cytosols of control or FLAG-tagged HDAC6-expressing cells prior to addition of the mixture to stripped GR immune pellets for heterocomplex reconstitution for 20 min at 30° C.

Assay of Steroid Binding Capacity—For cytosols to be assayed for steroid binding, a 50 μl aliquot of cytosol was incubated overnight at 4° C. in 50 μl HEM buffer plus 50 nM [³H]dexamethasone plus or minus a 1,000-fold excess of non-radioactive dexamethasone. Samples were mixed with dextran-coated charcoal, centrifuged, and counted by liquid scintillation spectrometry. The steroid binding is expressed as counts/min of [³H]dexamethasone bound/100 μl of cytosol.

Washed immune pellets to be assayed for steroid binding to stable GR.hsp90 heterocomplexes were incubated overnight at 4° C. in 50 μl HEM buffer plus 50 nM [³H]dexamethasone. Samples were then washed three times with 1 ml of TEGM buffer and counted by liquid scintillation spectrometry. For assay of steroid binding under dynamic GR.hsp90 assembly conditions, 50 nM [³H]dexamethasone was present during the assembly incubation at 30° C., and pellets were then washed and counted. In both cases, the steroid binding is expressed as counts/min of [³H]dexamethasone bound/FiGR immunopellet prepared from 100 μl of Sf9 cell cytosol.

Gel Electrophoresis and Western Blotting. Immune pellets were resolved on 12% SDS-polyacrylamide gels and transferred to Immobilon-P membranes. The membranes were probed with 0.25 μg/ml BuGR2 for GR, 1 μg/ml AC88 for hsp90, 1 μg/ml JJ3 for p23, 0.1% α-AcK, or 0.1% α-HDAC6. The immunoblots were then incubated a second time with the appropriate ¹²⁵I-conjugated or horseradish peroxidase-conjugated counterantibody to visualize the immunoreactive bands.

Protein Purification. Hsp70, Hop, YDJ-1 (the yeast homolog of hsp40) and p23 were purified as described by Kanelakis and Pratt (17). When hsp90 was purified from HDAC6 knockdown cells by the 3-step procedure (17), it was deacetylated and functionally identical to purified wild-type hsp90 in supporting stable GR.hsp90 heterocomplex assembly in the five-protein assembly system. Because hsp90 binds to ATP-agarose when the salt concentration of the application buffer is low and can then be eluted with a salt gradient, a single-step procedure of ATP-agarose chromatography was used, both to partially purify hsp90 and to compare the relative ATP-binding properties of hsp90 from knockdown and wild-type cells. This procedure rapidly separates hsp90 from deacetylating activity and yields acetylated hsp90 from knockdown cytosol that does not support stable GR.hsp90 heterocomplex assembly. For ATP affinity chromatography, 2.0 ml of cytosol prepared in HEM buffer was applied to a 50 ml column of ATP-agarose, the column was washed with 100 ml of HE buffer (10 mM Hepes, pH 7.4, 2 mM EDTA), and the column was then eluted with a 125 ml gradient of (0-500 mM) KCl in HE buffer. Hsp90 is eluted with the KCl gradient and the matrix is subsequently cleared of hsp70 and other high affinity ATP-binding proteins by elution with 5 mM ATP. The hsp90-containing fractions were identified by Western blotting, pooled, and contracted to 200-250 μl by Amicon filtration. It is important to freeze the preparation in multiple small aliquots and to unfreeze them only once.

GR-Mediated Transcriptional Activation. Dexamethasone-induced CAT gene expression was assayed by measuring CAT enzymatic activity in wild-type and knockdown cell cytosol, using a modified version of the CAT assay described in Kwok et 01. (18). Cells transfected with MMTV-CAT reporter and treated for 20 h with various concentrations of dexamethasone were washed, harvested, resuspended in potassium phosphate buffer (100 mM potassium phosphate, 1 mM dithiothreitol, pH 7.8), and ruptured by exposing the cell suspensions to three freeze-thaw cycles. Cell suspensions were centrifuged at 18,000×g for 10 min and protein concentration of the supernatants was measured by Bradford assay. Aliquots of the supernatants containing 10 μg total protein were incubated for 15 min at 70° C. in 150 mM Tris-HCI buffer, pH 7.4. The aliquots were added to a CAT reaction mixture (50 nM purified [³H]chloramphenicol, 150 mM Tris-HCl, pH 7.4, 0.25 mM butyryl CoA) and incubated for 2 h at 37° C. An organic phase mixture consisting of 2 parts pristane and 1 part mixed xylenes was added and samples were thoroughly vortexed. The reaction mixture was centrifuged at 20,000× g for 10 min and 150 μl of the organic phase was counted by liquid scintillation spectrometry.

Hsp90 Binding to GR and Steroid Binding Activity Are Decreased in HDAC6 Knockdown Cells—FK228, an inhibitor of multiple histone deacetylases, has been reported to deplete cells of several hsp90 client proteins (e.g. p53, ErbB2, Raf-1) (4). However, selective knockdown of HDAC6 in HEK 293T cells results in decreased glucocorticoid binding activity without any decrease in the level of endogenous human GR. To further examine the effect of HDAC6 knockdown, the OR was immunoadsorbed the GR and its activity in formation of GR.hsp90 heterocomplexes was directly examined. Because an immunoadsorbing antibody against the human GR was not available, the mouse GR was transiently expressed in HEK cells. Decreased steroid binding activity was observed in HDAC6 knockdown cells without any decrease in the level of expressed mGR. mGR was immunoadsorbed from both wild-type and HDAC6 knockdown cells and the immune pellets were immunoblotted to detect coadsorbed hsp90 and p23. Very little hsp90 or p23 was detected in mGR immune pellets from knockdown cells compared to wild-type cells.

The GR in HDAC6 Knockdown Cells Has Normal Ability to Form Complexes with Hsp90. The cochaperone p23 stabilizes GR.hsp90 complexes, both in cell-free assembly (19) and in vivo (20). Thus, it is possible that acetylation of p23 or of the GR itself could account for decreased steroid binding activity and decreased recovery of GR.hsp90 heterocomplexes from HDAC6 knockdown cells. However, no acetylation was detected of either the mGR or p23 immunoadsorbed from knockdown cells under conditions where acetylation of knockdown hsp90 could be visualized. To determine if the mGR from HDAC6 knockdown cells could form stable GR.hsp90 complexes, the mGR was incubated with rabbit reticulocyte lysate. The mGR from knockdown cells had the same ability to form mGR.rabbit hsp90 heterocomplexes with the same steroid binding activity as mGR from wild-type cells. Thus, the mGR from HDAC6 knockdown cells appears to be intrinsically normal and competent to become a functional receptor in the presence of wild-type hsp90 chaperone machinery.

HDAC6 Knockdown Cytosol Is Deficient at Stable GR.hsp90 Heterocomplex Assembly. To determine if HDAC6 knockdown cells were deficient at GR.hsp90 heterocomplex assembly, baculovirus-expressed mGR was immunoadsorbed from Sf9 cytosol, stripped of insect chaperones, and incubated with cytosols prepared from wild-type and knockdown cells. Cytosol from HDAC6 knockdown cells was shown to have reduced ability to form GR.hsp90 heterocomplexes and generate steroid binding activity.

In all cases where client proteins form heterocomplexes with hsp90 that are stable enough to survive immunoadsorption and washing, inhibition of hsp90 function (e.g., by geldanamycin) leads to degradation via the ubiquitylation/proteasome pathway (3). This is the case for the GR (21), and the level of GR in HDAC6 knockdown cells is the same as that in wild-type cells, despite the reduced ability of knockdown cells to form GR.hsp90 heterocomplexes. However, in some cases, hsp90 client proteins engage in a very dynamic cycle of heterocomplex assembly/disassembly, with disassembly being so rapid that no, or only trace amounts of, client protein.hsp90 heterocomplexes are observed with biochemical techniques. This is the case with nNOS, for example, which associates with hsp90 in a very dynamic manner that is sort of a “hit-and-run” mode of hsp90 regulation (22). However, such a dynamic cycle of heterocomplex assembly/disassembly nevertheless stabilizes nNOS to proteasomal degradation (23).

The GR undergoes a similar dynamic cycle of hsp90 heterocomplex assembly/disassembly in vitro when p23 is omitted from the purified assembly system (19). Such a dynamic assembly cycle can be detected by having radiolabeled dexamethasone present during the assembly incubation (24). The [³H]dexamethasone binds to the receptor as GR.hsp90 complexes are formed, thus steroid binding constitutes evidence that the chaperone machinery has carried out hsp90-dependent opening of the steroid binding cleft. Replicate GR immune pellets were incubated with cytosols from wild-type and HDAC6 knockdown 293T cells in the absence of dexamethasone and then washed and incubated with [³H]dexamethasone to detect stable heterocomplex assembly or they were incubated with cytosols in the presence of [³H]dexamethasone to detect dynamic heterocomplex assembly. Although the HDAC6 knockdown cytosol is deficient at stable GR.hsp90 heterocomplex assembly, it has the same activity as wild-type cytosol at dynamic heterocomplex assembly.

Purified Rabbit hsp90 Restores Stable Heterocomplex Assembly of HDAC6 Knockdown Cytosol to the Level of Wild-Type Cytosol. To determine if the decreased stable heterocomplex assembly activity of HDAC6 knockdown cytosol was due to altered function of hsp90, purified rabbit hsp90 was added to knockdown cytosol and GR.hsp90 heterocomplex assembly activity and steroid binding activity were assayed. Addition of purified hsp90 brings stable heterocomplex assembly activity and steroid binding activity up to the levels of wild-type cytosol. This indicates that components of the assembly machinery other than hsp90 are not affected by HDAC6 knockdown.

It has been reported that hsp90 immunoadsorbed from knockdown cells has much less p23 bound to it (15). Thus, it is possible that acetylation of hsp90 reduces its p23 binding affinity and that increasing the concentration of p2.3 could overcome this deficiency. It has also previously been shown that the stoichiometry of p23 to hsp90 in reticulocyte lysate is ˜1:9 and that p23 is the limiting component of the hsp90/hsp70-based chaperone system in lysate (20). When purified p23 is added to reticulocyte lysate to achieve approximate stoichiometric equivalence with hsp90, there is an increase in stable GR.hsp90 heterocomplex recovery and steroid binding activity (20). A similar increase in stable assembly is seen when purified p23 is added to wild-type 293T cytosol. Addition of purified p23 to HDAC6 knockdown cytosol yields the same percentage increase in stable assembly but it does not alter the deficiency in assembly with respect to wild-type cytosol. Thus, it seems unlikely that acetylation of hsp90 just reduces its affinity for p23, and it is likely that acetylation makes hsp90 unable to respond to p23 at all.

Purified hsp90 from HDAC6 Knockdown Cells Has Decreased ATP-binding Affinity. Cytosols prepared from HDAC6 knockdown 293T cells are deficient at stable GR.hsp90 heterocomplex assembly, but they nevertheless have 20 to 50% of the stable assembly activity of cytosols from wild-type cell. The ability to form some stable heterocomplexes suggests that there is a mixture of acetylated and deacetylated hsp90 in knockdown cytosol. Hsp90 was purified from knockdown cytosol using the 3-step protocol involving sequential chromatography on DEAE-cellulose, hydroxyapatite and ATP-agarose (17). The purified hsp90 was then assayed for GR.hsp90 heterocomplex assembly activity in a five-protein mixture containing purified rabbit hsp70, purified human Hop, purified human p23, and purified YDj-1, the yeast homolog of hsp40 (17). The purified HDAC6 knockdown cell hsp90 had the same activity at stable GR.hsp90 heterocomplex assembly as hsp90 purified from wild-type 293T cells. This suggested that the knockdown cell hsp90 was deacetylated during its purification, and partial deacetylation probably also occurred when knockdown cytosol was incubated at 30° C. during GR.hsp90 heterocomplex assembly.

HDAC6 knockdown or wild-type 293T cytosol prepared in low salt buffer was applied to a column of ATP-agarose and eluted with a gradient of 0-500 mM KCl. The hsp90-containing fractions identified by immunoblotting were pooled, contracted, and tested for both stable and dynamic GR.hsp90 assembly in the purified five-protein system. The HDAC6 knockdown cell hsp90 elutes from ATP-agarose at a low salt concentration and hsp90 from the wild-type cell elutes at high salt: This suggests that the acetylated hsp90 in knockdown cells has a lower ATP-binding affinity than the deacetylated hsp90 in wild-type cells. The hsp90 purified from HDAC6 knockdown cells has no stable GR.hsp90 heterocomplex assembly activity in the purified five-protein system, but it retains dynamic assembly activity.

Incubation with HDAC6 Restores Stable GR.hsp90 Heterocomplex Assembly Activity to Knockdown hsp90. It has been shown that immunopurified FLAG-HDAC6 deacetylates hsp90 whereas a catalytically dead HDAC6 mutant does not (15). The five-protein assembly mixture containing purified HDAC6 knockdown cell hsp90 was incubated with immunopurified FLAG-HDAC6 and stable GR.hsp90 heterocomplex assembly was assayed. The stable heterocomplex assembly activity of purified knockdown cell hsp90 is restored to the level of purified wild-type hsp90 by incubation with immunopurified FLAG-HDAC6. Incubation of wild-type hsp90 with FLAG-HDAC6 does not affect its ability to generate steroid binding.

The Dexamethasone Dose-Response Curve is Shifted to the Right ˜100 fold in HDAC6 Knockdown Cells. The GR contains a short 7-amino acid segment at the N-terminus of the ligand binding domain that is required for hsp90 binding and steroid binding activity (25). Mutations of three amino acids in this segment of the rat GR to alanine (P548A/V551A/S552A) yields a triple mutant GR that engages in dynamic GR.hsp90 heterocomplex assembly/disassembly in vivo (26). The dose-response curve for dexamethasone-dependent gene transactivation is shifted ˜300-fold to the right in cells expressing the triple mutant GR compared to the wild-type GR (26). Because GR.hsp90 heterocomplex assembly/disassembly is similarly dynamic in HDAC6 knockdown 293T cells, experiments were conducted to determine if they had the same phenotype with regard to the dose-response for dexamethasone. Wild-type or HDAC6 knockdown 293T cells transiently transfected with an MMTV-CAT reporter plasmid were treated for 20 h with various concentrations of dexamethasone prior to assay of CAT activity. The dose-response for dexamethasone-dependent CAT activity was shifted ˜100-fold to the right in HDAC6 knockdown versus wild-type cells. In addition to the right shift in the dose-response, there is a decrease in the maximal induction of CAT activity. As noted previously, the wild-type and HDAC6 knockdown cells have the same levels of GR protein, and it is not known why the maximal transactivating activity is decreased. The decrease may indicate an action of HDAC6 on proteins other than hsp90 (e.g., coactivator proteins) involved in the hormone response.

The studies described herein show that specific depletion of HDAC6 renders glucocorticoid receptors in HEK 293T cells deficient in steroid binding activity and in stable heterocomplex assembly. Neither the level of the GR nor its intrinsic ability to be assembled into stable GR.hsp90 heterocomplexes with steroid binding activity are affected by HDAC6 knockdown. Cytosol prepared from knockdown cells is deficient in its ability to assemble stable GR.hsp90 heterocomplexes, but the assembly activity is restored to the level of wild-type cytosol by addition of purified rabbit hsp90. This suggests that hsp90 is the only component of the multichaperone assembly machinery that is affected by depletion of HDAC6. Consistent with this, hsp90 purified from HDAC6 knockdown cells is deficient at stable GR.hsp90 heterocomplex assembly when it is the hsp90 component of a purified five-protein assembly system. The deficiency in stable assembly by hsp90 from knockdown cytosol is reversed by preincubating with HDAC6.

### Example III

### HDAC6 Regulates Hsp90 Client Proteins Raf-1 (C-Raf) and B-Raf

Raf kinase family member Raf-1 (C-Raf) and B-Raf are oncogenes in human cancer and both are Hsp90 client proteins critical for cell proliferation. Inactivation of Hsp90 results in the degradation of Raf-1 and B-Raf. This study shows that Raf-1 and B-Raf protein levels are reduced in cells deficient in HDAC6.

Mouse embryonic fibroblasts (MEF) derived from wild type (WT) or HDAC6 mutant embryo (KO) were analyzed for B-Raf and Raf-1 protein levels by specific antibodies. Both B-Raf and Raf-1 protein levels were observed to be down in HDAC6 deficient cells. In HDAC6 deficient mouse embryo fibroblasts, Raf-1 and B-Raf protein levels are both reduced compared to those in wild type cells.

Raf-1 and B-Raf protein levels can be restored to near wild type by re-constituting the HDAC6 deficient cells with a wild type HDAC6. Ectopically expressed HDAC6 restores B-Raf and Raf-1 protein levels in HDAC6 deficient cells. HDAC6 deficient MEFs were reconstituted with a plasmid encoding for wild type HDAC6 fused to GFP (GFP-HDAC6) via retrovirus mediated gene transfer. The Raf-1 and B-Raf levels were restored to almost wild type levels in the reconstituted lines. Actin protein levels were used as the loading control.

A similar reduction in B-Raf protein levels can be observed in human prostate cancer cells when HDAC6 is transiently inactivated via a siRNA specific for HDAC6. Transient knockdown of HDAC6 leads to reduced B-Raf protein levels in LNCAP. Prostate cancer cell line LNCAP were transfected with a control siRNA (WT) or HDAC6 specific siRNA (knockdown, KD) and the protein levels for B-Raf were analyzed. B-RAF levels were reduced in HDAC6 knockdown cells. This is similar to the effect of Hsp90 inhibitor geldanamycin (GA) treatment. Acetylated tubulin levels were induced in HDAC6 KD cells as shown previously (Hubbert et al. (2002) “HDAC6 is a microtubule-associated deacetylase” Nature 417:455-458.

These findings indicate that HDAC6 is required for maintaining normal Raf-1 and B-Raf protein levels, indicating that inactivation of HDAC6 could inhibit Raf-1 or B-Raf dependent oncogenesis. As Raf kinases operate upstream of ERK kinases, these findings are also in agreement with the observation that loss of HDAC6 impairs activation-associated phosphorylation of ERK kinases.

### Example IV

### HDAC6 Regulates Critical Hsp90 Co-Chaperones

Hsp90 molecular chaperone activity requires co-chaperones. Among those, p60^{HOP} and p50^{cdc37} have been implicated to be important in oncogenic transformation and kinase activation. The protein levels for p60^{HOP} and p50^{cdc37} in wild type (WT) and HDAC6 deficient MEFs (KO) were determined by specific antibodies. The results of these studies showed that inactivation of HDAC6 leads to the degradation of p60^{HOP} and p50^{cdc37}. A clear reduction of p60^{HOP} and p50^{cdc37} was observed in HDAC6 deficient MEF cells. Both protein levels were reduced in HDAC6 KO cells. CHIP, another Hsp90 co-chaperone was not affected by HDAC6.

HDAC6 was transiently inactivated in LNCAP by siRNA as described herein. Both p60^{HOP} and p50^{cdc37} levels were reduced in HDAC6 knockdown cells. Hsp90 levels were not affected by HDAC6

The reduction of p60^{HOP} and p50^{cdc37} can be partially rescued by a proteasome inhibitor MG132 treatment, indicating that loss of HDAC6 results in the degradation of p60^{HOP} and p50^{cdc37} in the proteasomes. Since p60^{HOP} and p50^{cdc37} are critical co-chaperones for Hsp90 function, these observations provide further evidence that HDAC6 is required for full Hsp90 chaperone function.

### Example V

### HDAC6 is Required for ErbB2-Induced Tumor Transformation

This study shows that inactivation of HDAC6 by siRNA markedly reverses the transformed phenotype of SKBR3, an ErbB2-overexpressing human breast cancer cell line and that ErbB2- induced transformation is suppressed in fibroblasts deficient in HDAC6. These observations demonstrate that HDAC6 is required for efficient ErbB-2-induced oncogenic transformation, thereby providing the rationale for targeting HDAC6 in treating breast cancer caused by overexpression of ErbB2. Similar studies will be carried out for other oncogenic kinases such as Src, BCR-ABL and AKT to demonstrate the utility of targeting HDAC6 in cancer therapies where these oncogenes are involved.

Experiments were conducted to demonstrate that anchorage independent growth is impaired in SKBR3 with loss of HDAC6. Whole cell lysates from a vector control and HDAC6 knockdown SKBR3 cells were immunoblotted for HDAC6 and total actin. HDAC6 knockdown SKBR3 cells showed a significantly lower level of HDAC6. Experiments were also conducted wherein fifty thousand SKBR3 cells stably expressing a siRNA for HDAC6 (SKBR3-HD6 KD) or vector control (SKBR-V) were plated in 0.3% soft agar with regular medium for three weeks. Colony formation in soft agar was quantitated, showing impairment of anchorage independent growth with loss of HDAC6.

Additional experiments demonstrated that full ErbB2-dependent oncogenic transformation requires HDAC6. ErbB2 is co-expressed with either dominant negative p53 of SV40 large T antigen in fibroblasts derived from wild type or HDAC6 deficient mouse embryos. These cells were then assayed for their ability to grow on soft agar as described above. Quantification of colony formation in soft agar showed reduced anchorage dependent growth in the absence of HDAC6.

### Example VI

### Inactivation of HDAC6 by Specific Deacetylase Inhibitor or siRNA Leads to ErbB2 and EGFR Degradation

This study demonstrates that loss of HDAC6 leads to accelerated EGER degradation. Control and HDAC6 knockdown (KD) A549 cells were treated with EGF for different times and the EGFR protein level was determined by immunoblotting. The relative EGFR level was quantitated over time, showing that the level of EGFR and its half-life were much reduced in HDAC6 knockdown cells.

Thus, the present invention is directed to therapies targeting EGFR and ErbB2 for cancers associated with these proteins, by attacking EGFR and ErbB2 through a novel mechanism mediated by HDAC6-regulated Hsp90 acetylation. The combined application of inhibitors of HDAC6 with other drugs targeting EGFR, such as kinase inhibitors, can be used to significantly improve tumor prognosis. Furthermore, as noted above, Hsp90 controls several critical oncogenic client protein kinases in addition to ErbB2 family members, including oncogenic src, AKT, BCR-ABL and dominant negative p53 and thus the development of specific approaches to inhibit Hsp90 through HDAC6 has broad utility in treating various types of tumors associated with different oncogenic kinases. In addition, as HDAC6 has been shown in mice to be a nonessential protein, the selective inactivation of Hsp90 by modulating its acetylation via HDAC6 inhibition offers a novel, more specific and less toxic cancer therapy.

Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference into this application in order to more fully describe the state of the art to which this invention pertains.

### REFERENCES

Cadepond et al. (1991) “Heat shock protein 90 as a critical factor in maintaining glucocorticosteroid receptor in a nonfunctional state” J Riot Chem 266:5834-5841.
Cohen and Yao. (2004) AcK-knowledge reversible acetylation. Sci STKE 2004, pe42.
Dittmar et al. (1997) “Folding of the glucocorticoid receptor by the heat shock protein (hsp) 90-based chaperone machinery. The role of p23 is to stabilize receptorthsp90 heterocomplexes formed by hsp90.p60.hsp70” J Biol Chem 272:21213-21220.
Furumai et al. (2001) “Potent histone deacetylase inhibitors built from trichostatin A and cyclic tetrapeptide antibiotics including trapoxin” Proc Natl Acad Sci USA 98:87-92.
Giguere et al. (1986) “Functional domains of the human glucocorticoid receptor” Cell 46:645-652.
Haggarty et al. (2003) “Domain-selective small-molecule inhibitor of histone deacetylase 6 (HDAC6)-mediated tubulin deacetylation” Proc Natl Acad Sci USA 100:4389-4394.
Hubbert et al. (2002) “HDAC6 is a microtubule-associated deacetylase” Nature 417:455-458.
Johnson and T oft. (1994) “A novel chaperone complex for steroid receptors involving heat shock proteins, immunophilins, and p23” J Biol Chem 269:24989-24993.
Kamal et al. (2003) “A high-affinity conformation of Hsp90 confers tumour selectivity on Hsp90 inhibitors” Nature 425:407-410.
Kawaguchi et al. (2003) The Deacetylase HDAC6 Regulates Aggresome Formation and Cell Viability in Response to Misfolded Protein Stress” Cell 115:727-738.
Komatsu et al. (2003) “Four different clones of mouse anti-acetyllysine monoclonal antibodies having different recognition properties share a common immunoglobulin framework structure” J Immunol Methods 272:161-175.
Matsuyama et al. (2002) “In vivo destabilization of dynamic microtubules by HDAC6-mediated deacetylation” EMBO J 21:6820-6831.
Morishima et al. (2003) “The hsp90 cochaperone p23 is the limiting component of the multiprotein hsp90/hsp70-based chaperone system in vivo where it acts to stabilize the client protein: hsp90 complex” J Biol Chem 278:48754-48763.
Neckers (2002) “Hsp90 inhibitors as novel cancer chemotherapeutic agents” Trends Mol Med 8:S55-61.
Nimmanapalli et al. (2003) “Histone deacetylase inhibitor LAQ824 both lowers expression and promotes proteasomal degradation of Bcr-Abl and induces apoptosis of imatinib mesylate-sensitive or -refractory chronic myelogenous leukemia-blast crisis cells” Cancer Res 63:5126-5135.
Pratt and Toft (2003) “Regulation of signaling protein function and trafficking by the hsp90/hsp70-based chaperone machinery” Exp Biol Med (Maywood) 228:111-133.
Richter and Buchner (2001) “Hsp90: chaperoning signal transduction. J Cell Physiol 188:281-290.
Schneider et al. (1996) “Pharmacologic shifting of a balance between protein refolding and degradation mediated by Hsp90” Proc Nati Acad Sci USA 93:14536-14541.
Verdel et al. (2000) “Active maintenance of mHDA2/mHDAC6 histone-deacetylase in the cytoplasm” Curr Biol 10:747-749.
Whitesell and Cook (1996) “Stable and specific binding of heat shock protein 90 by geldanamycin disrupts glucocorticoid receptor function in intact cells” Mol Endocrinol 10:705-712.
Xu et al. (2001) “Sensitivity of mature ErbB2 to geldanamycin is conferred by its kinase domain and is mediated by the chaperone protein Hsp90” J Biol Chem 276:3702-3708.
Yu et al. (2002) “Modulation of p53, ErbB1, ErbB2, and Raf-1 expression in lung cancer cells by depsipeptide FR901228” J Nail Cancer Inst 94:504-513.
Zhang et al. (2003) “HDAC-6 interacts with and deacetylates tubulin and microtubules in vivo” EMBO J 22:1168-1179.
3. Isaacs et al. (2003) “Heat shock protein 90 as a molecular target for cancer therapeutics” Cancer Cell 3:213-217
4. Yu et al. (2002) “Modulation of p53, ErbB1, ErbB2, and Raf-1 expression in lung cancer cells by depsipeptide FR901228” J Natl Cancer Inst 94:504-513.
7. Hubbert et al. (2002) “HDAC6 is a microtubule-associated deacetylase” Nature 417:455-458.
8. Kawaguchi et al. (2003) The Deacetylase HDAC6 Regulates Aggresome Formation and Cell Viability in Response to Misfolded Protein Stress” Cell 115:727-738.
15. Kovacs et al. (2005) “HDAC6 regulates hsp90 acetylation and chaperone-dependent activation of glucocorticoid receptor” Molecular Cell, 18:601-607
16. Morishima et al. (2000) “Stepwise assembly of a glucocorticoid receptor.sp90 heterocomplex resolves two sequential ATP-dependent events involving first hsp70 the hsp90 in opening of the steroid binding pocket” J. Biol. Chem. 275:18054-18060
17. Kanelakis and Pratt (2003) “Regulation of glucocorticoid receptor ligand-binding activity by the hsp70/hsp90-based chaperone machinery” Methods Enzymol. 364:159-173
18. Kwok et al. (1994) “Nuclear protein CBP is a coactivator for the transcription factor CREB” Nature 370:223-226
19. Dittmar et al. (1997) “Folding of the glucocorticoid receptor by the heat shock protein (hsp) 90-based chaperone machinery. The role of p23 is to stabilize receptor.hsp90 heterocomplexes formed by hsp90.p60.hsp70” J Biol Chem 272:21213-21220.
20. Morishima et al. (2003) “The hsp90 cochaperone p23 is the limiting component of the multiprotein hsp90/hsp70-based chaperone system in vivo where it acts to stabilize the client protein: hsp90 complex” J Biol Chem 278:48754-48763.
21. Whitesell and Cook (1996) “Stable and specific binding of heat shock protein 90 by geldanamycin disrupts glucocorticoid receptor function in intact cells” Mol Endocrinol 10:705-712.
22. Billecke et al. (2004) “The role of hsp90 in heme-dependent activation of apo-neuronal nitric-oxide synthase” J. Biol. Chem. 279:30252-30258
23. Bender et al. (1999) “Neuronal nitric-oxide synthase is regulated by the hsp90-based chaperone system in vivo” J. Biol. Chem. 274:1472-1478
24. Dittmar and Pratt (1997) “Folding of the glucocorticoid receptor by the reconstituted hsp90-based chaperone machinery” J. Biol. Chem. 272:13047-13054
25. Xu et al. (1998) “Binding of hsp90 to the glucocorticoid receptor requires a specific 7-amino acid sequence at the amino terminus of the hormone-binding domain” J. Biol. Chem. 273:13918-13924
26. Kaul et al. (2002) “Mutations at positions 547-553 of rat glucocorticoid receptors reveal that hsp90 binding requires the presence, but not defined composition, of a seven-amino acid sequence at the amino terminus of the ligand binding domain” J Biol Chem 277:36223-36232

What is claimed is:

1. A method of inhibiting Hsp90 activity in a cell, comprising contacting the cell with an inhibitor of histone deacetylase 6 (HDAC6) activity.

2. A method of treating a cancer associated with Hsp90 in a subject, comprising administering to the subject an effective amount of an inhibitor of HDAC6 activity.

3. The method of claim 2, wherein the cancer is a cancer associated with an oncoprotein selected from the group consisting of ErbB2, EGFR, AKT, BCR-abl, src, C-Raf B-Raf, dominant negative p53, HIF-1α, Telomerase, MTG8 (myeloid leukemia protein), Heat Shock factor and Hepatitis B virus reverse transcriptase.

4. A method of modulating steroid receptor signaling in a cell, comprising contacting the cell with an inhibitor of HDAC6 activity.

5. The method of claim 4, wherein the steroid receptor is selected from the group consisting of a glucocorticoid receptor, an androgen receptor an estrogen receptor, a progesterone receptor and a mineralocorticoid receptor.

6. A method of treating a disorder associated with aberrant steroid receptor signaling in a subject, comprising administering to the subject an effective amount of an inhibitor of HDAC6.

7. The method of claim 6, wherein the disorder is cancer, muscle atrophy, type II diabetes, polycystic ovarian syndrome, male pattern baldness, uterine fibroids and endometriosis.

8. The method of claim 1, wherein the cell is in a subject.

9. The method of claim 2, wherein the subject is human.

10. The method of claim 4, wherein the cell is in a subject.

11. The method of claim 6, wherein the subject is a human.

12. The method of claim 1, wherein the inhibitor of HDAC6 activity is selected from the group consisting of hydroxamic acid based HDAC inhibitors, Suberoylanilide hydroxamic acid (SAHA) and its derivatives, NVP-LAQ824, Trichostatin A, Scriptaid, m-Carboxycinnamic acid bishydroxamic acid (CBHA), ABHA, Pyroxamide, Propenamides, Oxamflatin, 6-(3-Chlorophenylureido)caproic hydroxamic acid (3-C1-UCHA), A-161906, jnj16241199, tubacin and tubacin analogs, siRNA, short chain fatty acid HDAC inhibitors, butyrate, phenylbutyrate, valproate, hydroxamic acid, trichostatins, epoxyketone-containing cyclic tetrapeptides, HC-toxin, Chlamydocin, Diheteropeptide, WF-3161, Cyl-1, Cyl-2, non-epoxyketone-containing cyclic tetrapeptides, Apicidin, cyclic-hydroxamic-acid-containing peptides (CHAPS), benzamides and benzamide analogs, CI-994, deprudecin, organosulfur compounds and any combination thereof.
